(19) United States
(12) Patent Application Publication
(10) Pub. No.: US 2010/0190212 A1
(43) Pub. Date: Jul. 29, 2010

(54) METHOD OF CONTROLLING THE CONTENT OF SELECTED COMPONENT(S) FROM POLYMER(S) USING MOLECULAR SIEVE(S)

(75) Inventors: Eric Thwaites, Copenhagen (DK); Martin Barfoed, Espergaerde (DK); Poul Bach, Birkerod (DK)

Correspondence Address: NOVOZYMES NORTH AMERICA, INC., 500 FIFTH AVENUE, SUITE 1600, NEW YORK, NY, 10110, US

(73) Assignee: Novozymes Biopolymer A/S, Bagsvaerd (DK)

(21) Appl. No.: 12/753,962

(22) Filed: Apr. 5, 2010

Related U.S. Application Data
(63) Continuation of application No. 11/416,691, filed on May 2, 2006.
(60) Provisional application No. 60/679,300, filed on May 10, 2005.

(30) Foreign Application Priority Data
May 4, 2005 (DK) PA 2005 00661

(51) Int. Cl. C12P 19/28

(52) U.S. Cl. 435/85

(57) ABSTRACT

The invention provides in a first aspect a method for controlling the content of selected component(s) in one or more polymer(s) by:
(a) contacting the polymer(s) with at least one molecular sieve; and optionally
(b) isolating the polymer from the molecular sieve(s).

### CROSS-REFERENCE TO RELATED APPLICATIONS

This application is a continuation of U.S. application Ser. No. 11/416,691 filed on May 2, 2006 which claims the benefit of Danish Application No. PA 2005 00661 filed May 4, 2005 and U.S. Provisional Application No. 60/679,300 filed May 10, 2005, which applications are incorporated herein by reference.

### FIELD OF THE INVENTION

The present invention relates to a method for controlling the content of selected component(s) in one or more polymer(s).

### BACKGROUND OF THE INVENTION

Polymers and their derivatives are used in a wide range of applications. Of particular interest are biopolymers which can be applied in the food, cosmetic, medical and pharmaceutical industries. In order to meet these diverse applications it is often necessary to purify the polymer to remove unwanted components and, further, to control the balance of components in the final product.

The most abundant heteropolysaccharides of the body are the glycosaminoglycans. Glycosaminoglycans are unbranched carbohydrate polymers, consisting of repeating disaccharide units (only keratan sulphate is branched in the core region of the carbohydrate). The disaccharide units generally comprise, as a first saccharide unit, one of two modified sugars —N-acetylgalactosamine (GalNAc) or N-acetylglucosamine (GlcNAc). The second unit is usually an uronic acid, such as glucuronic acid (GlcUA) or iduronate.

Glycosaminoglycans are negatively charged molecules, and have an extended conformation that imparts high viscosity when in solution. Glycosaminoglycans are located primarily on the surface of cells or in the extracellular matrix. Glycosaminoglycans also have low compressibility in solution and, as a result, are ideal as a physiological lubricating fluid, e.g., joints. The rigidity of glycosaminoglycans provides structural integrity to cells and provides passageways between cells, allowing for cell migration. The glycosaminoglycans of highest physiological importance are hyaluronan, chondroitin sulfate, heparin, heparan sulfate, dermatan sulfate, and keratan sulfate. Most glycosaminoglycans bind covalently to a proteoglycan core protein through specific oligosaccharide structures. Hyaluronan forms large aggregates with certain proteoglycans, but is exceptional as free carbohydrate chains form non-covalent complexes with proteoglycans.

Numerous roles of hyaluronan in the body have been identified (see, Laurent and Fraser, 1992, FASEB J. 6: 2397-2404; and Toole B. P., 1991, “Proteoglycans and hyaluronan in morphogenesis and differentiation.” In: Cell Biology of the Extracellular Matrix, pp. 305-341, Hay E. D., ed., Plenum, N.Y.). Hyaluronan is present in hyaline cartilage, synovial joint fluid, and skin tissue, both dermis and epidermis. Hyaluronan is also suspected of having a role in numerous physiological functions, such as adhesion, development, cell motility, cancer, angiogenesis, and wound healing. Due to the unique physical and biological properties of hyaluronan, it is employed in eye and joint surgery and is being evaluated in other medical procedures.

HA plays an important role in the biological organism, as a mechanical support for the cells of many tissues, such as the skin, tendons, muscles and cartilage, it is a main component of the intercellular matrix. HA also plays other important parts in the biological processes, such as the moistening of tissues, and lubrication.

HA may be extracted from the above mentioned natural tissues, although today it is preferred to prepare it by microbiological methods to minimize the potential risk of transferring infectious agents, and to increase product uniformity, quality and availability.

HA and its various molecular size fractions and the respective salts thereof have been used as medicaments, especially in treatment of arthropathies, as an auxiliary and/or substitute agent for natural organs and tissues, especially in ophtalmology and cosmetic surgery, and as agents in cosmetic preparations. Products of hyaluronan have also been developed for use in orthopedics, rheumatology, and dermatology.

HA may also be used as an additive for various polymeric materials used for sanitary and surgical articles, such as polyurethanes, polyesters etc. with the effect of rendering these materials biocompatible.

Due to the wide diversity of biopolymer usage particularly HA usage and derivatives thereof, some of which are mentioned above, and the frequent use of HA in pharmaceutical compositions or surgical articles as well as tailored for specific applications, it is often necessary to provide HA products of high purity which should be substantially absent of other contaminating components in the end product. The non-polymer content and ionic composition of the polymer, particularly of HA, is also important. Often, the sodium salt of the HA is preferred as the most biocompatible form and other HA salts (Fe, Ca, Cu, Zn, Al, Mg, Mn) are avoided, especially Ca++ salts of HA. Although, in certain applications, it can be desirable to favour another salt of HA or create a controlled balance of counter ions. For example, controlled levels of calcium are actually desirable in some wound care applications, controlled zinc levels have been proposed for combating foot ulcers (Diabetologia Croatica 30-3, 2001) and for antibacterial properties (Acta Pharm Hung., 2002, 72(1): 15-24); and controlled iron levels are sometimes used to control the rheological properties in some types of hyaluronic acid gel (CN 1473572A; WO 95/04132), whereas, low iron levels are often desired to reduce HA polymer susceptibility to degradation.

Conventionally polymers, including HA, having predominantly the sodium salt form and low or no Ca++ and low or no other metal ion content have been provided by carefully avoiding the presence of calcium and other unwanted ions during the fermentation process step and during subsequent purification steps. The desired ion balance is conventionally achieved by first creating an environment of high sodium ion concentration from, for example, addition of sodium salt(s) such as sodium acetate, sodium chloride, sodium sulphate, etc. The high sodium ion concentration is used to competitively displace calcium from the HA molecule. The calcium ions liberated from the HA molecule can then be removed from the HA molecule, or vice versa, by any of a wide range of conventional polymer isolation processes. Most commonly, the polymer, or particularly HA, is precipitated or crystallised chemically and/or by organic solvent addition, such as ethanol, iso-propylalcohol, acetone, chloroform, CETAB etc, thereby leaving the liberated and unwanted ions in the supernatant (CZ 9700350; WO 84/03302; EP 0694616 and EP 0144019 being just some examples of this process). The liberated ions can also be separated from the polymer by ultra-filtration of dia-filtration techniques (WO 95/04132; GB 2249315). It is also possible to use aqueous extraction and other common polymer separation techniques.

Other methods to displace undesirable components, often Ca++, from a polymer include sequestration with sequestration agents, such as, EDTA, phosphates (CN 85103674), etc or application ion exchange adsorbent resins (EP 0694616).

The disadvantages inherent in the techniques described above, depending on the chosen method and the active agent(s) used, are that: repeated application is often required to achieve the desired component balance (e.g. repeated precipitation and re-suspension or extensive dia-filtration); and or the component selectivity is poor; and or the capacity for the component is poor; and or the displacement or sequestration efficiency is low; and or process introduces another component which is subsequently difficult to remove and/or is toxic; and or process stream conditions need to be manipulated to achieve the desired component balance.

Accordingly alternative processes for manipulating the balance of components associated with a polymer is desirable, due to the difficulties described above for the conventional means of controlling the component balance in the final polymer product. The present invention provides such a process, which furthermore, provides several advantages as will be described.

### SUMMARY OF THE INVENTION

The invention provides in a first aspect a method for controlling the content of selected component(s) in one or more polymer(s) by:

(a) contacting the polymer(s) with at least one molecular sieve; and optionally

(b) isolating the polymer from the molecular sieve(s).

### DETAILED DESCRIPTION OF THE INVENTION

The present invention provides a method for controlling the content of undesirable or selected components from a polymer by removing or replacing the component(s) with a more desirable component and or manipulating the balance of components associated with the polymer. The removal or manipulation of the component(s) is performed by contacting the polymer product with an appropriate molecular sieve(s) for a time period sufficient to remove or exchange or manipulate the component balance. In some applications it can be convenient to leave the molecular sieve, in with the polymer product; however, if desirable, the molecular sieve(s) may be separated from the polymer.

The term “polymer” in the present context is a substance which is made up of many repeating smaller chemical units or molecules. Polymers can be natural or synthetic.

The term “polymer” also comprises liquid polymers or suspensions of polymers, in this context.

The “component” to be removed or manipulated comprises atoms, molecules, ions, or compounds.

In the present context the expression “control” means remove (completely or partially), exchange and/or balance the content of component(s) from the polymer or polymer bearing liquid.

There are numerous applications where it is desirable to control, reduce or select the components associated with the polymer and/or polymer bearing liquid:

i) For example, for biocompatibility, hyaluronic acid is preferred in the sodium form. Furthermore, limitations for specific applications are often desired, for example, calcium containing polymers may be insoluble themselves, (e.g. calcium alginate) or may create problematic precipitation in common phosphate buffers, or create adverse reactions with active ingredients or formulation chemicals. Conversely, a controlled level of calcium in some applications has been specified in, for example, alginate for wound care products. Similarly, Fe, Ca, Cu, Zn, Al, Mg, other ions and other components

ii) Ionic content and ion type can affect the viscosity and other properties of the polymer; similarly for other components. Ion content has to be carefully and accurately controlled, for example, for the creation of hydrogels containing zinc.

iii) Polymer destabilising molecules, such as Fe, and Cu can be removed, controlled or replaced by another less harmful ion.

iv) Polymer products often require removal of odour or colour molecules.

The present invention provides a number of advantages over conventionally applied methods for manipulation of the component balance. The molecular sieve(s) according to the invention can be applied at any point in the manufacturing process, including to raw materials; during manufacture or as a post-treatment of the polymer product. Furthermore the process conditions such as pH, temperature, polymer concentration etc. are not as critical as in other conventionally applied processes.

Molecular sieves can be highly selective for particular components or groups of components. Reaction equilibrium is normally achieved rapidly meaning faster processing and closer control of the product characteristics in terms of the component balance associated with the polymer. The molecular sieves demonstrate a high capacity for the component(s) to be manipulated and therefore there is generally only the need for a single molecular sieve(s) treatment. The simple addition, contact and subsequent removal of the molecular sieve(s) by conventional solid-liquid separation methods, means there is no need for dedicated equipment and no need to subsequently remove soluble reactants or additions. It is possible to leave the molecular sieve(s) in the polymer solution which further simplifies the process. Process costs can also be reduced since the present method allows raw materials containing components which are undesirable in the end product, to be used in upstream steps, e.g. tap water (Ca⁺⁺ containing) for fermentation and dilution instead of de-ionized water. Furthermore the molecular sieve(s) can often be added directly and without the requirement for pre-equilibrium or pre-treatment. Molecular sieves are generally low in toxicity.

### High Molecular Weight Contaminants/Impurities

Since the method according to the invention is applied for the removal of component(s) from a polymer product, component(s) may even be added in an upstream process step without causing any problems for the downstream purification steps.

Since the method according to the invention is conveniently applied for the removal of calcium from a polymer product, Ca++ may even be added in an upstream process step without causing any problems for the downstream purification steps.

The addition of calcium, or other divalent salts, makes it possible to remove high molecular weight contaminants/impurities by flocculation in an early purification step; it is also possible to remove these impurities from cell free preparations of the glycosaminoglycan of interest. This is further described in WO 2004/001054. The possibility of adding component(s), particularly calcium during upstream process steps constitutes a further advantage of the present invention compared to traditional methods.

The above advantages are provided by the method according to the invention for controlling the content of selected (often undesirable) components from one or more polymers comprising the steps of:

(a) contacting the polymer(s) with a molecular sieve; and optionally

(b) isolating the polymer product from the molecular sieve(s).

In the present context “controlling the content of selected components” means removing (completely or partially) the component(s) and or replacing (exchanging) the component(s) with a more desirable component and or manipulating the balance of components associated with the polymer or polymer bearing liquid.

A “molecular sieve” in the present context means materials having molecule-sized pores that can be used in separating larger molecules from smaller ones. They include, but are not limited to, zeolites, carbon molecular sieves, silica gels, activated alumina. Typically, molecular sieves have a lattice structure creating a cage like structure with windows which admit only molecules of less than a certain size. By using different source materials and different conditions of manufacture, it is possible to produce a range of molecular sieves of differing access dimensions. The dimensions can often be precise for a particular molecular sieve(s) because they derive from the crystal structure of that sieve.

Examples of some of the molecules admitted by different molecular sieves are given in Table 17.3 “Classification of Some Molecular Sieves” from Chemical Engineering, Volume 2, 4^{th} Edition; J M Coulson and J F Richardson; Pergamon Press, 1991 together with further details on molecular sieves. A more comprehensive database of relevant structures and the properties of molecular sieves is hosted by the International Zeolite Association (www.iza-online.org/) at (www.iza-structure.org/databases/). Relevant basic texts for molecular sieves include: D W Breck: Zeolite Molecular Sieves, Wiley, New York, 1974; R M Barrer: Hydrothermal Chemistry of Zeolites, Academic Press, 1982; Intro to Zeolite Science and Practice, H van Bekkum, E M Flannigen, P A Jacobs and J C Jansen, Studies in Surface Science, Vol 137, 1-1060, (2001) Elsevier, Amsterdam.

In a particular embodiment the molecular sieve(s) is a zeolite. The classical definition of a zeolite is a crystalline, porous aluminosilicate. However, some relatively recent discoveries of materials virtually identical to the classical zeolite, but consisting of oxide structures with elements other than silicon and aluminium have stretched the definition. Most researchers now include virtually all types of porous oxide structures that have well-defined pore structures due to a high degree of crystallinity in their definition of a zeolite. In the present context all of the above are comprised in the term “zeolite”. In these crystalline materials we call zeolites, the metal atoms (classically, silicon or aluminum) are surrounded by four oxygen anions to form an approximate tetrahedron consisting of a metal cation at the centre and oxygen anions at the four apexes. The tetrahedral metals are called T-atoms for short, and these tetrahedra then stack in, regular arrays such that channels form. The possible ways for the stacking to occur is virtually limitless, and hundreds of unique structures are known.

The zeolitic channels (or pores) are microscopically small, and in fact, have molecular size dimensions such that they are often termed “molecular sieves”. The size and shape of the channels have extraordinary effects on the properties of these materials for adsorption processes, and this property leads to their use in separation processes. Components can be separated via shape and size effects related to their possible orientation in the pore, and or by differences in strength of adsorption. Therefore, components can be selectively removed.

Since silicon typically exits in a 4+ oxidation state, the silicon-oxygen tetrahedra are electrically neutral. However, in zeolites, aluminium typically exists in the 3+ oxidation state; so that aluminium-oxygen tetrahedra form centres that are electrically deficient one electron. Thus, zeolite frameworks are typically anionic, and charge compensating cations populate the pores to maintain electrical neutrality.

In a particular embodiment of the invention the molecular sieve is chosen from the group of zeolites where the porosity of the material is compatible with the component to be removed, in a further embodiment the porosity of the material is compatible with Ca++; in a further embodiment ions populating the zeolite pores to maintain electrical neutrality are those desired in the polymer product; in a further embodiment sodium ions are the ions populating the zeolite pores to maintain electrical neutrality.

Those molecular sieves classically grouped as “Type 4” have molecular sieve(s) dimensions appropriate for the sequestration of calcium ions having Linde sieve 4A dimensions of approximately 0.4 nm. A number of these molecular sieves have sodium ions populating the zeolite pores to maintain electrical neutrality.

In a particular embodiment of the invention the zeolite is therefore a Type 4 zeolite. In a further embodiment the pores of the molecular sieve contains sodium ions.

In one embodiment of the invention the polymer is a biopolymer. A “biopolymer” is any polymeric substance (examples being, but not limited to, polysaccharides, proteins nucleic acids, etc,) formed in a biological system. Many examples of common biopolymers exist, including, but not limited to: Chitosan, glucan, keratin, cellulose, gelatine, glycosaminoglycans and derivatives of all these polymers.

In a particular embodiment the biopolymer is a polysaccharide, and in a further particular embodiment the polysaccharide is a glycosaminoglycan.

### Glycosaminoglycans

According to the invention a glycosaminoglycan may be any carbohydrate polymer having a molecular weight of at least 700 Daltons; preferably a molecular weight of at least 10,000 Daltons; more preferably a molecular weight of at least 20,000 Daltons, even more preferably a molecular weight of at least 30,000 Daltons.

Preferred glycosaminoglycans are hyaluronic acid, chondroitin sulphate, chondroitin (non-sulphated), heparin, heparin sulphate, dermatan sulphate, and keratin sulphate. Hyaluronic acid is constituted by alternating and repeating units of D-glucoronic acid and N-acetyl-D-glucosamine, to form a linear chain having a molecular weight of up to 15,000,000 Daltons.

Preferred Glycosaminoglycans according to the invention are Glycosaminoglycans having a molecular weight of from 700 Daltons to 15,000,000 Daltons.

It is to be noted that the term “hyaluronic acid” in the present application and claims may mean indifferently hyaluronic acid in its acidic form or in its salt form such as for example sodium hyaluronate, potassium hyaluronate, magnesium hyaluronate, calcium hyaluronate, or others.

The terms “hyaluronan” or “hyaluronic acid” are used in literature to mean acidic polysaccharides with different molecular weights constituted by residues of D-glucuronic and N-acetyl-D-glucosamine acids, which occur naturally in cell surfaces, in the basic extracellular substances of the connective tissue of vertebrates, in the synovial fluid of the joints, in the endobulbar fluid of the eye, in human umbilical cord tissue and in cocks' combs.

The term “hyaluronic acid” is in fact usually used as meaning a whole series of polysaccharides with alternating residues of D-glucuronic and N-acetyl-D-glucosamine acids with varying molecular weights or even the degraded fractions of the same, and it would therefore seem more correct to use the plural term of “hyaluronic acids”. The singular term will, however, be used all the same in this description; in addition, the abbreviation “HA” will frequently be used in place of this collective term.

The biopolymer, e.g., a glucosaminoglucan, can be provided from animal tissues or more preferably by culturing a host cell expressing the biopolymer.

### Fermentation Broth

The glycosaminoglycan may be obtained from any fermentation broth. The glycosaminoglycan may furthermore be one which is producible by a method comprising cultivating a host cell.

The host cell may preferably be a micro-organism. The micro-organism may be a unicellular micro-organism, e.g., a prokaryote, or a non-unicellular micro-organism, e.g., a eukaryote. Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus lichenifonnis, Bacillus megaterium, Bacillus pumilus, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a particular embodiment, the bacterial host cell is a Bacillus lentus cell, a Bacillus lichenifonnis cell, a Bacillus stearothermophilus cell or a Bacillus subtilis cell. Mutant Bacillus subtilis cells particularly adapted for recombinant expression are described in WO 98/22598.

The host cell may be a eukaryote, such as a mammalian cell, an insect cell, a plant cell or a fungal cell. Useful mammalian cells include Chinese hamster ovary (CHO) cells, HeLa cells, baby hamster kidney (BHK) cells, COS cells, or any number of other immortalized cell lines available, e.g., from the American Type Culture Collection. The transformation method, selectable marker gene and any other parts of the expression construct may be chosen from those well known and available to one skilled in the art.

The host cell may be a fungal cell. “Fungi” as used herein includes the phyla Ascomycota, Basidiomycota, Chytridiomycota, and Zygomycota as well as the Oomycota and all mitosporic fungi. Representative groups of Ascomycota include, e.g., Neurospora, Eupenicillium (=Penicillium), Emericella (=Aspergillus), Eurotium (=Aspergillus), and the true yeasts listed below. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g., Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

The fungal host cell may be a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g., genera Kluyveromyces, Pichia, and Saccharomyces). The basidiosporogenous yeasts include the genera Leucosporidim, Rhodosporidium, Sporidiobolus, Filobasidium, and Filobasidiella. Yeast belonging to the Fungi Imperfecti are divided into two families, Sporobolomycetaceae (e.g., genera Sporobolomyces and Bullera) and Cryptococcaceae (e.g., genus Candida).

In another embodiment, the fungal host cell is a filamentous fungal cell. “Filamentous fungi” include all filamentous forms of the subdivision Eumycota and Oomycota. The filamentous fungi are characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. In a more particular embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, and Trichoderma.

Fungal cells may be transformed by a process involving protoplast formation, transformation of the protoplasts, and regeneration of the cell wall in a manner known per se.

The micro-organism producing the glycosaminoglycan of interest is cultivated in a nutrient medium suitable for production of the glycosaminoglycan using methods known in the art. For example, the micro-organism may be cultivated by shake flask cultivation, small-scale or large-scale fermentation (including but not limited to continuous, batch, fed-batch, or solid state fermentations) in laboratory or industrial fermentors. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). As an example the production of a glycosaminoglycan produced in a micro-organism WO 2003/054163 describes the production of hyaluronic acid in a Bacillus host cell.

As mentioned above, traditional methods or processes for the production of purified glycosaminoglycans with a desired component balance, such as, e.g., hyaluronic acid in the sodium salt form, include:
Precipitation of the polymer in a sodium ion rich environment;
Crystallisation in a sodium ion rich environment;
Precipitation of the unwanted component, for example, with phosphate and application of a sodium ion rich environment;
Dialysis or Dia-filtration in a sodium ion rich environment;
Sequestration and application of a sodium rich environment; and
Other conventional means for polymer purification.

One problem associated with the known methods for removing calcium is that they almost always introduce a soluble contaminant that needs to be removed by further processing steps. Furthermore these methods often utilises chemical precipitants or other aids which are then subsequently difficult to remove from the product and may be toxic or detrimental to the final application. This is true of e.g. competitive substitution of the calcium in the presence of an excess of sodium ions. The displaced calcium and excess of sodium counter ions need to be removed. It is also the case when using sequestration agents such as EDTA. The EDTA must be subsequently removed. Also precipitation of the calcium ion, with say phosphates requires the excess of precipitant to be removed as well as the precipitate formed.

Such processing steps are less easy to control because the reaction equilibrium changes as the concentration of the soluble(s) components change, achievement of reaction equilibrium is often relatively slow, capacity for the component to be manipulated is often relatively low, the techniques only work for a narrow range of process conditions (e.g., pH, ionic strength, polymer concentration, etc). Furthermore they are often time consuming and require more than one application, which leaves the product susceptible to degradation and are not selective or efficient means to control the component balance in the product.

According to the present invention a method/process is provided comprising the steps of contacting the polymer or polymer bearing liquid to be modified with the appropriate type(s) of molecular sieve(s) under appropriate conditions; and separating the molecular sieve(s from the polymer, if necessary.

Contact between the polymer bearing liquid to be modified with the molecular sieve(s, can be achieved by any of a number of means including, but not limited to:
Simple suspension or mixing together
Passage of the polymer bearing liquid through a: packed, settled, expanded, fluidised bed of molecular sieves
Contact with molecular sieves used as a body feed, pre-coat or aid to filtration.

The type, amount and contacting conditions of the molecular sieve(s) required to achieve the desired component balance in the polymer product can be simply determined by someone skilled in the art.

The component to be removed or manipulated according to the invention comprises atoms, molecules, ions, or compounds. Examples of components which can be removed by particular molecular sieve types include, but are not limited to, those given in Table 17.3 “Classification of Some Molecular Sieves” from Chemical Engineering, Volume 2, 4^{th} Edition; J M Coulson and J F Richardson; Pergamon Press, 1991. A more comprehensive database of relevant structures and the properties of molecular sieves is hosted by the International Zeolite Association (www.iza-online.org/) at (www.iza-structure.org/databases/). Relevant basic texts for molecular sieves include:
D W Breck: Zeolite Molecular Sieves, Wiley, New York, 1974
R M Barrer: Hydrothermal Chemistry of Zeolites, Academic Press, 1982
Intro to Zeolite Science and Practice, H van Bekkum, E M Flannigen, P A Jacobs and J C Jansen, Studies in Surface Science, Vol. 137, 1-1060, (2001) Elsevier, Amsterdam

Particularly, zeolites can be used to control the content of (remove or manipulate) organic solvents (including but not limited to: alcohols, aldehydes, ketones, etc.), metal ions, anions, quaternary ammonium compounds, SDS, EDTA, CETAB, TCA, cetyl pyrimidine chloride, etc.

Particularly the molecular sieve(s) can be used to remove or manipulate the balance of ion, more particularly cations.

In one embodiment the ion is a divalent ion, more particularly Ca⁺⁺.

In a particular embodiment the invention relates to a method for controlling the content of calcium ions from a polymer using a molecular sieve(s).

In a particular embodiment the invention relates to a method for controlling the content of calcium and sodium ions from a polymer using a molecular sieve(s).

The component(s) to be removed or partly removed can be suspended in the liquid comprising the polymer or the component(s) can be associated or be present on the polymer.

In a further embodiment the component(s) is exchanged for another component. Ca-ions can e.g. be exchanged for Na-ions.

In a still further embodiment the component(s) balance in the product is controlled. In a particular embodiment the molecular sieve(s) is a zeolite.

“Appropriate conditions” in this context mean those of the process stream and those conditions which can be easily determined by one skilled in the art for effecting the component removal or manipulation. The appropriate conditions can include but are not limited to; molecular sieve type, molecular sieve dosing, temperature, pH, polymer concentration, ionic strength, solvent concentration, mixing, incubation time, etc.

In a more particular embodiment the polymer is a glycosaminoglycan.

The Molecular sieve(s) can be removed from the polymer by any of a number of means, for example, but not limited to: filtration, centrifugation, floatation, sedimentation, phase exclusion, etc. In some cases, it may not be necessary to remove, or may be desirable to leave, the Molecular sieve(s) in the polymer bearing liquid.

The particular component removal or manipulation process according to the invention can be optimised or improved through, for example, but not limited to, manipulation of: pH, temperature, viscosity, concentration, mixing, ionic strength, additives, ingredients, etc. More than one polymer may be treated in the same polymer bearing liquid. More than one type of Molecular sieve(s) may be contacted with the polymer bearing liquid. More than one treatment with Molecular sieve(s) may be used to manipulate the polymer(s) bearing liquid.

In a particular embodiment the method of the invention provides control (removal, exchange and/or balancing) of ions in the polymer bearing liquid using an appropriate zeolite. Particularly the polymer is a glycosaminoglycan.

In a more particular embodiment the polymer is hyaluronic acid.

In another particular embodiment the process of the invention provides control of the content of ions on the polymer itself using an appropriate zeolite. Particularly the polymer is hyaluronic acid.

In still another embodiment the process of the invention provides control of the content of calcium and sodium ions on the polymer itself using an appropriate molecular sieve. Particularly the polymer is hyaluronic acid.

### EXAMPLES

### Example 1

### Hyaluronic Acid Purification without a Calcium Removal Stage

In this experiment a 5 g/l solution of hyalyronic acid, obtained from fermentation of a recombinant Bacillus subtilis, was diluted with ordinary tap water and filtered to remove host cells. The filtrate was then extensively dialysed against deionised water to remove the bulk of free calcium not on the HA molecule itself. The resulting dialysed product contained 4.4 wt. % calcium relative to the mass of hyaluronic acid.

### Example 2

### Hyaluronic Acid Purification with a Conventional Calcium Removal Stage by Dia-filtration Against a Sodium Salt

In this experiment hyaluronic acid was obtained as described in Experiment 1. A calcium controlling step (not involving molecular sieve(s) according to the invention) was introduced involving competitive substitution of the calcium in the presence of an excess of sodium ions as follows:

The hyaluronic acid solution was dia-filtered against an excess of sodium ions. The liberated calcium ions were thereby removed from the solution by passage through the dia-filtration membrane. The liberated calcium ions from the hyaluronic acid could equally have been removed by polymer precipitation. In the present case the hyaluronic acid solution was dia-filtered against 3× volumes of a 10 wt. % sodium sulphate solution at constant volume followed by extensive dialysis against deionised water to remove excess sulphate and sodium ions. The resulting product contained 1.2 wt. % calcium relative to the hyaluronic acid.

### Example 3

### Control of Calcium Content of Hyaluronic Acid Using a Cation Exchange Resin

Hyaluronic acid was produced as described in Example 1 above. A strong cation exchange resin with SO₃⁻(>2 eq/l) in the sodium ion form was used to manipulate the calcium ion content of the polymer.

The performance of the exchange resin was characterised for the range of conditions likely to be met throughout hyaluronic acid manufacturing and purification processes described earlier.

In one example, 1.0 wt. % exchange resin was added to a 0.5 wt. % solution of hyaluronic acid containing 2 wt. % calcium relative to the hyaluronic acid. After 120 minutes of incubation with stirring the resin was filtered from the solution. The resulting filtrate contained hyaluronic acid with a calcium content of 0.5 wt. % calcium relative to the hyaluronic acid. After an incubation of 240 minutes under the same conditions the calcium content relative to the hyaluronic acid was below detection.

### Example 4

### Control of Calcium Content of Hyaluronic Acid Using a Cation Exchange Gel

Hyaluronic acid was produced as described in Example 1 above. A strong cation exchange gel having a sulphonated functional group (2.05 eq/l) was used to manipulate the calcium ion content of the polymer.

The performance of the exchange resin was characterised for the range of conditions likely to be met throughout hyaluronic acid manufacturing and purification processes described earlier.

In one example, 0.25 wt. % exchange gel was added to a 0.5 wt. % solution of hyaluronic acid containing 2 wt. % calcium relative to the hyaluronic acid. The mixture was incubated with stirring and allowed to come to equilibrium which was achieved in 1 hour. The gel was filtered from the solution. The resulting filtrate contained hyaluronic acid with a calcium content of 0.3 wt. % calcium relative to the hyaluronic acid. Under the same conditions, 1 wt. % of the exchange gel in the sodium form reduced the calcium content relative to the hyaluronic acid to below detection.

In another pilot scale example, 0.5 wt. % exchange gel in the sodium form was added, without pre-treatment, to a 0.7 wt. % solution of hyaluronic acid containing 211 ppm calcium. The mixture was incubated with stirring for 2 hours before the gel was filtered from the solution. The resulting filtrate contained 14 ppm calcium relative to the hyaluronic acid corresponding closely to bench scale experiments under the same conditions. Under the same conditions, 1 wt. % of the exchange gel reduced the calcium content relative to the hyaluronic acid to below detection.

The starting material and the filtrate treated with 1 wt. % of the exchange gel in the sodium form were dialysed against deionised water to remove free ions. From analysis of the resulting hyaluronic acid, it was found that the ions on the hyaluronic acid, following treatment with 1 wt. % of the exchange gel in the sodium form, had been replaced by sodium ions.

### Example 5

### Reduction of Iron in Fermentation Broth Using a Cation Exchange Gel

Gel type was a strong cation exchanger having functional groups: sulphonates (2.05 eq/l).

An excess of the ion exchange gel was used to reduce the level of iron in the fermentation broth. Ions such as iron, copper and others have been demonstrated to reduce the stability of hyaluronic acid towards degradation.

Raw fermentation broth containing hyaluronic acid was clarified by dilution with ordinary tap water and filtration to remove the microorganisms. This clarified broth was then incubated and stirred with an excess (10 wt. %) of exchange gel for 1 hour. The exchange gel was then filtered from the solution. The iron content of the filtrate was found to be below detection (<1 ppm relative to the hyaluronic acid) compared to 0.4 wt. % in the clarified broth (relative to the hyaluronic acid)

The original clarified broth and that treated to remove iron were subsequently heat treated. The exchange gel treated material was found to be substantially more heat stable with regard to molecular weight than the untreated clarified broth material under the same conditions.

Treatment of the clarified broth did not change the molecular weight profile or concentration of the hyaluronic acid contained.

It was found that other process fluids and components could be controlled with similar application and procedures to those demonstrated in experiments 3 and 4. Removal of components from the clarified fermentation broth stabilised the hyaluronic acid towards thermal degradation.

### Example 6

### Control of Calcium Content of Hyaluronic Acid and Replacement of Ions on the Hyaluronic Acid with Sodium Ions Using the Sodium Form of Powdered Aluminium Silicate Type 4A Zeolite

Hyaluronic acid was produced as described in Example 1 above. A powdered Type 4A zeolite in the sodium form was used to manipulate the calcium ion content of the polymer.

The performance of the exchange resin was characterised for the range of conditions likely to be met throughout hyaluronic acid manufacturing and purification processes described earlier. The characteristics were reproduced at both bench and pilot scale and diverse hyaluronic acid batches, produced as in Experiment 1, were tested.

The zeolite was found to be able to remove around 0.06 mg calcium from the hyaluronic acid bearing solutions per mg zeolite when the two were contacted. This ratio was found to be largely independent of the process conditions used (eg pH, temperature, HA concentration, etc). This made it very simple to control the calcium level in the hyaluronic acid bearing solution by simple dosed addition of zeolite. Equilibrium was achieved in less than 15 minutes in all cases.

In a typical example only 0.2 wt. % of powdered zeolite was contacted with a hyaluronic acid solution containing 142 ppm calcium by direct addition without pre-equilibriation or pre-treatment. After 20 minutes incubation with stirring, the zeolite was filtered from the solution. The resulting filtrate contained 14 ppm calcium. Contact with 0.3 wt. % zeolite under the same procedure reduced the calcium to below detection limits.

Hyaluronic acid starting material and after treatment with 0.3 wt. % of the powdered zeolite were analysed. It was found that the ions on the hyaluronic acid, following treatment with 0.3 wt. % of the zeolite, had been replaced by sodium ions.

Treatment of the hyaluronic acid did not change the molecular weight profile or concentration and a detailed characterisation of the treated hyaluronic acid revealed no detrimental changes.

It was found that the degree of calcium removal from the hyaluronic acid containing solution could be reproducibly predicted and controlled. The ions on the hyaluronic acid were replaced by sodium ions. There were no adverse effects on the hyaluronic acid.

### Example 7

### Reduction of Iron in Fermentation Broth Using the Sodium Form of Powdered Aluminium Silicate Type 4A Zeolite

Ions such as iron, copper and others have been demonstrated to reduce the stability of hyaluronic acid towards degradation. An excess of the sodium form of a powdered aluminium silicate Type 4A Zeolite was used to reduce the level of iron in fermentation broth.

Raw fermentation broth obtained from fermentation of a recombinant Bacillus subtilis, was diluted with ordinary tap water and filtered to remove host cells to give a 3.5 g/l hyaluronic acid solution containing 24 ppm iron ion. This clarified broth was then incubated and stirred with an excess (3 wt. %) of powdered zeolite for 1 hour. The zeolite was then filtered from the solution. The iron content of the filtrate was found to be below detection (<1 ppm relative to the hyaluronic acid).

The original clarified broth and that treated to remove iron were subsequently heat treated. The exchange gel treated material was found to be substantially more heat stable with regard to molecular weight than the untreated clarified broth material under the same conditions.

Treatment of the clarified broth did not change the molecular weight profile or concentration of the hyaluronic acid contained.

### Example 8

### Control of Calcium Content of Hyaluronic Acid and Replacement of Ions on the Hyaluronic Acid with Sodium Ions Using a Sodium Form of a Granular Type 4A Zeolite

The performance of the granular zeolite for calcium ion removal and replacement with sodium ion was characterised for the range of conditions likely to be met throughout hyaluronic acid manufacturing and purification processes described earlier. The characteristics were reproduced at both bench and pilot scale and diverse hyaluronic acid batches, produced as in Experiment 1, were tested.

Typically, this granular Type 4A zeolite was able to remove around 0.1 mg calcium from the hyaluronic acid bearing solutions per mg zeolite when the two were contacted. This ratio was found to be largely independent of the process conditions used. This made it very simple to control the calcium level in the hyaluronic acid bearing solution. Equilibrium was achieved in less than 10 minutes in all cases.

In a typical example 0.2 wt. % of the granular zeolite was contacted with a hyaluronic acid solution containing 220 ppm calcium. After 20 minutes incubation with stirring, the zeolite was filtered from the solution. The resulting filtrate contained 20 ppm calcium. Contact with 0.25 wt. % zeolite (II) under the same conditions reduced the calcium to below detection limits.

Hyaluronic acid from the starting material and from the filtrate treated with 0.25 wt. % of the granular zeolite was analysed. It was found that the ions on the hyaluronic acid, following treatment with 0.25 wt. % of the zeolite had been replaced by sodium ions.

Treatment of the hyaluronic acid did not change the molecular weight profile or concentration and a detailed characterisation of the treated hyaluronic acid revealed no detrimental changes.

It was found that the degree of calcium removal from the hyaluronic acid containing solution could be reproducibly predicted and controlled. The ions on the hyaluronic acid were replaced by sodium ions. There were no adverse effects on the hyaluronic acid.

1-20. (canceled)

21. A method for producing a hyaluronic acid, comprising:
(a) culturing a microorganism to form a fermentation medium comprising the hyaluronic acid; and
(b) contacting the fermentation medium with a molecular sieve(s) comprising a Type 4 zeolite to control the content of selected component(s), wherein
the contacting is provided by (i) suspension or mixing the fermentation medium and molecular sieve(s) together, (ii) passage of the fermentation medium through a packed, settled, expanded, fluidized bed of molecular sieve(s), or (iii) contacting the fermentation medium with molecular sieve(s) used as a body feed, pre-coat or aid to filtration; and
the fermentation medium after the contacting is more heat stable with regard to molecular weight of the hyaluronic acid than the fermentation medium before the contacting.

22. The method of claim 21, wherein the component(s) are selected from the group consisting of atoms, molecules, ions, and compounds.

23. The method of claim 22, wherein the component(s) are an ion.

24. The method of claim 23, wherein the ion is a divalent ion.

25. The method of claim 24, wherein the divalent ion is Ca⁺⁺.

26. The method of claim 21, wherein the component(s) is removed or partially removed from the fermentation medium.

27. The method of claim 21, wherein the component(s) is removed or partially removed from the hyaluronic acid.

28. The method of claim 21, wherein the component(s) is exchanged for another component.

29. The method of claim 25, wherein the Ca⁺⁺ is exchanged for another component.

30. The method of claim 29, wherein the Ca⁺⁺ is exchanged for sodium.

31. The method of claim 30, wherein the Ca⁺⁺ is exchanged for sodium and the ratio of calcium form to sodium form of the hyaluronic acid is controlled.

32. The method of claim 21, wherein the pores of the molecular sieve(s) contain sodium ions.

33. The method of claim 21, further comprising isolating the hyaluronic acid from the molecular sieve(s).

34. The method of claim 21, wherein the microorganism is a Bacillus cell.

35. The method of claim 21, wherein the Bacillus cell is a Bacillus lentus cell, a Bacillus lichenifonnis cell, a Bacillus stearothermophilus cell or a Bacillus subtilis cell.
